(19) United States
(12) Patent Application Publication
(10) Pub. No.: US 2020/0121228 A1
(43) Pub. Date: Apr. 23, 2020

(54) BILIRUBIN ESTIMATION USING SCLERA COLOR AND ACCESSORIES THEREFOR

(71) Applicant: University of Washington, Seattle, WA (US)

(72) Inventors: James A. Taylor, Seattle, WA (US); Shwetak N. Patel, Seattle, WA (US); Alex T. Mariakakis, Seattle, WA (US)

(73) Assignee: University of Washington, Seattle, WA (US)

(21) Appl. No.: 16/617,469

(22) PCT Filed: Jun. 1, 2018
(86) PCT No.: PCT/US2018/035708

§ 371 Date: Nov. 26, 2019

Related U.S. Application Data
(60) Provisional application No. 62/513,825, filed on Jun. 1, 2017.

(51) Int. Cl. A61B 5/1455; A61B 5/00; A61B 5/103; A61B 5/145

(52) U.S. Cl. CPC A61B 5/1455; A61B 5/6803; A61B 2576/02; A61B 5/14546; A61B 2560/0233; A61B 5/1032

(57) ABSTRACT

Examples of systems and methods described herein may estimate the bilirubin level of an adult subject based on image data associated with a portion of the eye of the subject (e.g., a color of the sclera). Accessories are described which may facilitate bilirubin estimation, including sensor shields and calibration frames.

### CROSS-REFERENCE TO RELATED APPLICATION(S)

This application claims the benefit under 35 U.S.C. § 119 of the earlier filing date of U.S. Provisional Application Ser. No. 62/513,825 filed Jun. 1, 2017, the entire contents of which are hereby incorporated by reference in their entirety for any purpose.

### TECHNICAL FIELD

Examples described herein generally relate to bilirubin monitoring using image data of a subject's eye.

### BACKGROUND

The clinical gold standard for measuring bilirubin is through a blood draw called a total serum bilirubin (TSB). TSBs are invasive, require access to a healthcare professional, and are inconvenient if done routinely, such as for screening. An option for a non-contact alternative to a TSB for bilirubin is the transcutaneous bilirubinometer (TcB). A TcB shines a wavelength of light that is specifically reflected by bilirubin onto the skin and measures the intensity that is reflected back to the device.

### SUMMARY

Examples of methods are described herein. An example method includes extracting portions of image data associated with sclera from image data associated with an eye of a subject, generating features describing color of the sclera, and analyzing the features using a regression model to provide a bilirubin estimate for the subject.

Some example methods may include capturing the image data associated with the eye using a smartphone camera.

Some example methods may include positioning the smartphone camera over an aperture of a sensor shield, the sensor shield having at least one additional aperture positioned over the eye.

Some examples may include extracting portions of image data associated with sclera from image data associated with an eye of a subject at least in part by identifying a region of interest containing the sclera using pixel offsets associated with a geometry of the sensor shield.

Some examples may include capturing calibration image data in addition to the image data associated with the eye, the calibration image data associated with portions of frames worn proximate the eye.

Some examples may include extracting portions of image data associated with sclera from image data associated with an eye of a subject at least in part by identifying a region of interest containing the sclera by identifying the portions of image data within the frames.

Some examples may include color calibrating the image data.

In some examples, said color calibrating may include color calibrating with respect to portions of the image data containing known color values.

In some examples, generating features may include evaluating a metric over multiple pixel selections within the portions of image data.

In some examples, the metric may include median pixel value.

In some examples, generating features may include evaluating the metric over multiple color spaces of the portions of image data.

In some examples, generating features includes calculating a ratio between channels in at least one of the multiple color spaces.

In some examples, the regression model uses random forest regression.

Some examples may include initiating or adjusting a medication dose, or initiating or adjusting a treatment regimen, or combinations thereof, based on the bilirubin estimate.

Examples of systems are described herein. An example system may include a camera system including an image sensor and a flash, a sensor shield having a first aperture configured to receive the camera system and at least one second aperture configured to open toward an eye of a subject, the sensor shield configured to block at least a portion of ambient light from an environment in which the subject is positioned from the image sensor, and a computer system in communication with the camera system, the computer system configured to receive image data from the image sensor and estimate a bilirubin level of the subject at least in part by being configured to segment the image data to extract a portion of the image data associated with a sclera of the eye, generate features representative of a color of the sclera, and analyze the features using a machine learning model to provide an estimate of the bilirubin level.

In some examples, the camera system includes a smartphone and the sensor shield includes a slot configured to receive the smartphone and position the smartphone such that the image sensor and the flash of the smartphone are positioned at the first aperture.

In some examples, the sensor shield includes a neutral density filter and diffuser positioned between the first aperture and the at least one second aperture.

An example system may include calibration frames configured to be worn by a subject, the calibration frames configured to surround at least one eye of the subject when worn by the subject, the calibration frames comprising multiple regions of known colors, a camera system including an image sensor and a flash, the camera system configured to generate image data from the image sensor responsive to illumination of the at least one eye of the subject and the calibration frames with the flash, and a computer system in communication with the camera system, the computer system configured to receive the image data and estimate a bilirubin level of the subject at least in part by being configured to segment the image data to extract a portion of the image data associated with a sclera of the at least one eye, calibrate the portion of the image data in accordance with another portion of the image data associated with the calibration frames to provide calibrated image data, generate features representative of a color of the sclera using the calibrated image data, and analyze the features using a machine learning model to provide the estimate of the bilirubin level.

In some examples, the computer system is further configured to segment the image data at least in part based on a location of the calibration frames in the image data.

In some examples, the calibration frames comprise eyewear frames.

### BRIEF DESCRIPTION OF THE DRAWINGS

FIG. 1 is a schematic illustration of a system arranged in accordance with examples described herein.

FIG. 2 is a sequence of images illustrating methods of segmenting image data to identify regions associated with sclera arranged in accordance with examples described herein.

FIG. 3 is a schematic illustration of a sensor shield arranged in accordance with examples described herein.

FIG. 4 is a schematic illustration of calibration frames arranged in accordance with examples described herein.

FIG. 5 is a block diagram of example computing network arranged in accordance with an example embodiment.

FIG. 6 is a block diagram of an example computing device arranged in accordance with examples described herein.

### DETAILED DESCRIPTION

Certain details are set forth herein to provide an understanding of described embodiments of technology. However, other examples may be practiced without various of these particular details. In some instances, well-known circuits, control signals, timing protocols, and/or software operations have not been shown in detail in order to avoid unnecessarily obscuring the described embodiments. Other embodiments may be utilized, and other changes may be made, without departing from the spirit or scope of the subject matter presented here.

Several diseases may cause jaundice in subjects (e.g., patients). Jaundice may be manifested as a yellow discoloration of skin and sclera of the eye(s), which may be due to the buildup of bilirubin in the blood. Jaundice may only be recognizable to the naked eye in severe stages, but examples of systems and methods described herein may allow for a ubiquitous test using computer vision and/or machine learning which may be able to detect milder forms of jaundice. By detecting milder jaundice, patients and/or care providers may be alerted to the possibility of disease earlier, and may provide earlier interventions to halt or slow progression of a disease. Moreover, early detection of jaundice may allow for improved monitoring of surgical procedures or other interventions which may cause jaundice as a complication of the intervention. Examples described herein may be implemented using a smartphone application that captures pictures of one or more eyes of a subject and produces an estimate of the subject's bilirubin level, even at levels normally undetectable by the human eye. Two accessories are described which may improve operation of the system: (1) a sensor shield which may control the eyes' exposure to light and (2) calibration frames with colored areas for use in calibrating image data.

In an implemented example, an example system utilized with a sensor shield achieved a Pearson correlation coefficient of 0.89 and a mean error of −0.09±2.76 mg/di in predicting a person's bilirubin level. As a screening tool, the implemented example system detected cases of concern with a sensitivity of 89.7% and a specificity of 96.8% with the sensor shield.

Recall TcBs may be a non-invasive alternative to blood testing for jaundice. However, the computations underlying TcBs are generally designed for newborns, and their results simply do not translate correctly for adults. This may be because normal concentrations of bilirubin are much lower in adults compared to newborns (e.g., <1.3 mg/di vs. <15.0 mg/di). As it so happens, the sclera of the eye may be more sensitive than the skin to changes in bilirubin, which may be because their elastin has a high affinity for bilirubin. Accordingly, early, non-invasive screening may be provided by analysis of the sclera. Examples described herein accordingly may estimate the extent of jaundice in a person's eyes (e.g., estimate a bilirubin level) using image data taken from a computer system (e.g., a smartphone).

Generally, jaundice may not be apparent to a trained naked eye until bilirubin levels reach 3.0 mg/di, however, bilirubin levels as low as 1.3 mg/di may warrant clinical concern. Accordingly, there exists a detection gap between 1.3 and 3.0 mg/di that is missed by clinicians unless a TSB is requested, which is rarely done without due cause. Accordingly, systems as described herein that may quickly and conveniently provide an estimated bilirubin level may aid in screening individuals and catching cases of clinical concern.

Moreover, the trend of a person's bilirubin level over time may be more informative in some examples than just a single point measurement. If a person's bilirubin exceeds normal levels for one measurement but then returns to normal levels, it could be attributed to normal variation. If, however, a person's bilirubin shows an upward trend after it exceeds normal levels, it may be more likely that a pathologic issue is worsening their condition, such as a cancerous obstruction around the common bile duct. Trends may be not only important for diagnosis, but also for determining the effectiveness of treatment. One course of action for those affected by pancreatic cancer is the insertion of a stent in the common bile duct. The stent opens the duct so that compounds like bilirubin can be broken down again; a person's bilirubin level should decrease thereafter. If their bilirubin continues to rise, then there may be issues with the stent or the treatment may be ineffective. Trends in bilirubin levels are difficult to capture because repeated blood draws can be uncomfortable and inconvenient for many people, especially those in an outpatient setting. Examples of systems described herein may facilitate tracking of trends of bilirubin levels using convenient screening, which may aid in the monitoring of treatment efficacy.

Generally, an example system described herein may utilize a smartphone. The smartphone's built-in camera may be used to collect pictures of a person's eyes. The sclera, or white part of the eyes, may be extracted from the image using computer vision techniques. Features describing the color of the sclera may be produced and may be analyzed by a regression model to provide a bilirubin estimate. Since different lighting conditions can change the colors of the same scene, two accessories are described which may be used, together or separately, in some examples. The first accessory is a sensor shield, which may be a head-worn box. The sensor shield may simultaneously block out and/or reduce ambient lighting and provide controlled internal lighting through the camera's flash. A second accessory is calibration frames, which may be a pair of paper glasses printed with colored squares that facilitate calibration.

Accordingly, examples described herein may provide systems for convenient bilirubin testing with a variety of methods used for color calibration. Examples described herein may utilize a sclera segmentation methodology that may perform adequately for individuals with jaundice. Examples described herein may utilize machine learning models that relate the color of the sclera to a measure of bilirubin in the blood (e.g., a bilirubin level).

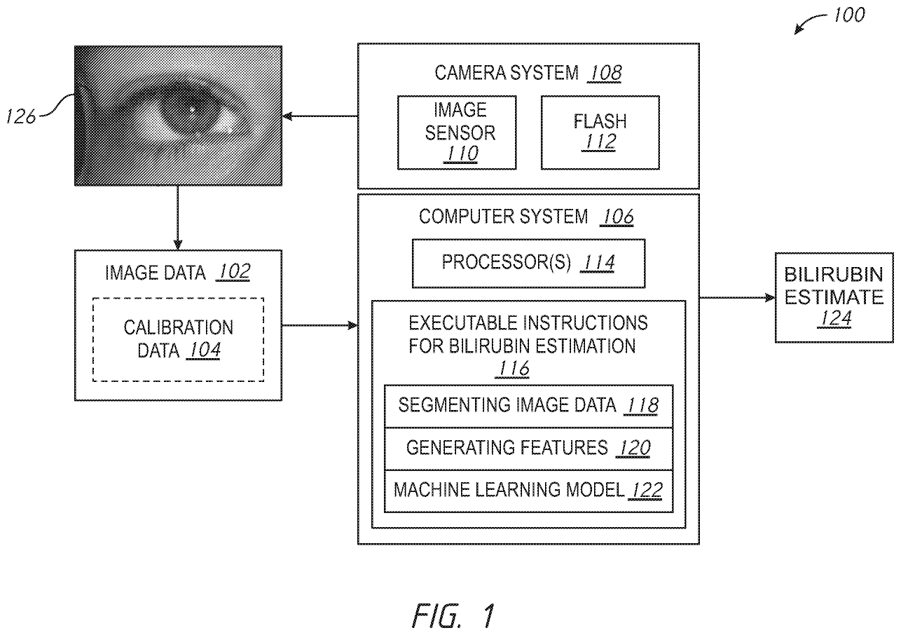

FIG. 1 is a schematic illustration of a system arranged in accordance with examples described herein. The system 100 includes camera system 108 and computer system 106. The camera system 108 may include image sensor 110 and flash 112. The computer system 106 may include processor(s) 114 and executable instructions for bilirubin estimation 116. The executable instructions for bilirubin estimation 116 may include instructions for segmenting image data 118, generating features 120, and machine learning model 122. The system 100 may utilize the camera system 108 to capture an image of eye 126 and provide image data 102. In some examples, the image data 102 may include calibration data 104 (e.g., from calibration frames or other calibrators placed proximate eye 126). The computer system 106 may receive the image data 102 and may analyze the image data 102 to provide bilirubin estimate 124.

While not explicitly shown in FIG. 1, the system 100 may include calibration frames, other calibration structures, and/or sensor shields. Additional, fewer, and/or different components may be included in other examples.

Systems described herein may include camera systems, such as camera system 108 of FIG. 1. While shown as a separate system from computer system 106 in FIG. 1, in some examples, the camera system 108 and computer system 106 may be integrated with one another (e.g., the camera system 108 and computer system 106 may be implemented as a smartphone, tablet, and/or computer). Camera system 108 may be implemented, for example, using a camera of a smartphone, tablet, and/or computer.

The camera system 108 includes image sensor 110. Any of a variety of image sensors may be used, which may generally include one or more charge-coupled devices (CCDs), photodiodes and/or other radiation-sensitive electronics. The image sensor 110 may generally provide an electrical signal responsive to incident radiation (e.g., light). In some examples, the image sensor 110 may include an array of image sensors, and may generate multiple pixels of image data responsive to incident radiation.

The camera system 108 includes flash 112. A flash 112 may not be used in other examples. The flash 112 may illuminate a subject to increase and/or manipulate an amount or kind of radiation reflected from a subject toward the image sensor 110. The flash 112 may be implemented using, for example, one or more light emitting diodes (LEDs) or other light sources. In some examples the flash 112 may be implemented using a white (e.g., broad spectrum) light source, however in some examples, the flash 112 may be implemented using one or more sources having a particular light spectrum (e.g., red, blue).

Examples of systems described herein may include one or more computing systems, such as computer system 106 of FIG. 1. While illustrated as a single computer system in FIG. 1, in some examples multiple computer systems may be in communication with one another and may provide the functions of the computer system 106 which is illustrated as a single system in FIG. 1. The computer system 106 may be implemented using, for example, one or more smartphones, computers, servers, desktops, laptops, tablets, appliances, or medical devices.

During operation, the camera system 108 may be used to generate image data 102, which may be representative of all or a portion of one or both eyes of a subject. For example, the camera system 108 may generate image data 102 from the image sensor 110 responsive to illumination of eye 126 with flash 112. In some examples, the flash 112 may illuminate eye 126 and a calibration structure (e.g., calibration frames) and the image data 102 may include calibration data 104 based on the calibration frames. Any of a variety of subjects may be used including humans (e.g., adults, children), and/or animals. Generally, the subject may be an entity having a bilirubin level that is of interest to a user of the system 100. A subject's eye may have a variety of portions, including a pupil, an iris, and a sclera. The sclera generally refers to connective tissue of an eyeball which typically may appear a particular color (e.g., white) in healthy subjects. Note that the eyeball may additionally have conjunctiva (e.g., a mucous membrane covering all or a portion of the eyeball, including the sclera). Examples described herein may refer to segmenting portions of image data relating to a subject's eye and estimating bilirubin levels of the subject based on a color of the sclera. It is to be understood that examples described herein refer to the color of the sclera region of images, which may include color contributed by the sclera and color contributed by the conjunctiva—for the purposes of examples described herein, no assumption may be made regarding whether a color change of the eye (e.g., yellowing) may occur in the actual sclera structure and/or the conjunctiva covering the sclera.

The computer system 106 includes processor(s) 114. The processor(s) 114 may be implemented, for example, using one or more processors, such as one or more central processing unit(s) (CPUs), graphical processing unit(s) (GPUs), including multi-core processors in some examples. In some examples, the processor(s) 114 may be implemented using customized circuitry and/or processing units, such as processing hardware specialized for machine learning or artificial intelligence computations, including, but not limited to, application specific integrated circuits (ASICs), and/or field programmable gate arrays (FPGAs). The computer system 106 may be configured to provide bilirubin estimates for a subject based on images of the subject's sclera. For example, the computer system 106 may be programmed to provide bilirubin estimates for the subject based on the image data 102. The computer system 106 may, for example, use one or more machine learning models to generate a bilirubin estimate based on the image data 102.

Accordingly, computer systems described herein may include software, such as executable instructions for bilirubin estimation 116. The executable instructions for bilirubin estimation 116 may include instructions for segmenting image data 118, generating features 120, and/or machine learning model 122. The executable instructions for bilirubin estimation 116 may be stored in one or more computer-readable media (e.g., memory, such as random access memory (RAM), read only memory (ROM) and/or storage, such as one or more disk drives, solid state drives, etc.). While not shown, the executable instructions for bilirubin estimation 116 may include instructions for color calibrating the image data in some examples.

The computer system 106 may include a variety of additional and/or different components, including but not limited to, communication devices and/or interfaces (e.g., wireless and/or wired communication) and input/output devices (e.g., one or more keyboards, displays, mice, touchscreens).

Examples of computer systems may accordingly segment image data (e.g., the computer system 106 of FIG. 1 may segment image data 102 using executable instructions for segmenting image data 118). Segmenting image data 102 generally refers to extracting portions of the image data 102 associated with portions of interest, such as sclera of a subject's eye. It is the portions of the image data 102 which are associated with the sclera which may be used by models described herein to estimate bilirubin levels.

Examples of computer systems may accordingly take the segmented portions of image data (e.g., the portions of image data pertaining to one or more sclera of a subject's eye(s)) and generate features describing the color of the sclera—e.g. in accordance with executable instructions for generating features 120. Features refer to values which may be representative of the sclera color (e.g., sclera and conjunctiva) based on the portions of the image data associated with the sclera. In some examples, features may be generated based on the image data (and/or the color calibrated image data). The features generally refer to one or more numerical metrics which may be calculated based on the image data and which may be representative of sclera color. In some examples, features are used which may correlate well with bilirubin level using a machine learning model. In some examples, generating features includes evaluating a metric over multiple pixel selections within the portions of image data associated with the sclera. Any of a variety of metrics may be used. In some examples, the metric may be a median pixel value. In some examples, metrics may be calculated using the image data represented in a variety of color spaces (e.g., RGB). Multiple sets of metrics may be calculated, one set for each color space in some examples. Accordingly, a metric may be evaluated over multiple color spaces. In some examples, one or more features may include a ratio between different color channels in one or more of the color spaces. Note that typically sclera color may be race- and/or age-agnostic. The normal color of sclera may be similar regardless of race and/or gender. Accordingly, it may not be necessary in some examples to utilize different and/or adjusted features based on race and/or gender—this is in contrast to methods which may utilize skin color as an indicator of jaundice, for example.

Examples of computer systems may take the generated features and provide a bilirubin estimate using those features—e.g., in accordance with executable instructions for using a machine learning model 122. For example, the features may be analyzed using a regression model to provide a bilirubin estimate. The bilirubin estimate may be an estimate of the bilirubin level in the subject's blood. The model (e.g., machine learning model 122) may be trained using ground truth data associating images of subject's eyes and their bilirubin level measured using other mechanisms (e.g., blood tests). In some examples, a random forest regression may be used. The bilirubin estimate may include a value for estimated bilirubin. The value may be between 0 and 5 mg/dl in some examples, between 0 and 4 mg/dl in some examples, between 0 and 3 mg/di in some examples, between 0 and 2 mg/dl in some examples. In some examples, instead of or in addition to providing an estimated value of bilirubin the blood stream, the bilirubin estimate may be an indication of whether the bilirubin level of the subject is within or outside of a normal range. The normal range may be less than 1.3 mg/dl in some examples, less than 1.2 mg/dl in some examples, less than 1.4 mg/dl in some examples, and other thresholds for normal may be used in other examples. In some examples, typical normal adult bilirubin levels may be around 0.6 mg/dl. In some examples, a normal adult bilirubin level may be considered to be a level less than 1.3 mg/dl, a borderline adult bilirubin level may be considered to be between 1.3 and 3.0 mg/dl, and an elevated (e.g., abnormal) adult bilirubin level may be considered to be greater than 3.0 mg/dl. Note that examples described herein which may provide bilirubin level estimates for adults may utilize higher precision than methods which may provide estimates of bilirubin level in newborns. This may because newborn bilirubin level may range over a generally wider range than adult bilirubin level (e.g., between 0 and 15 mg/di in newborns vs. between 0 and 3 mg/di in adults). Machine learning models may be used to provide a particular estimated bilirubin level in accordance with image data of one or more sclera and/or the machine learning models may be used to provide a screening indication (e.g., “normal” or “abnormal” and/or “normal”, “borderline”, or “abnormal”) in accordance with the image data.

While not explicitly shown in FIG. 1, in some examples, a sensor shield may be provided to block some or all of the ambient light present in the environment from the image sensor 110 and/or the eye 126. For example, a box having an aperture to receive the camera system 108 and another aperture to receive the eye 126 (e.g., be positioned over the eye 126) may be used. In some examples, the sensor shield may include or be coupled to a slot for holding the camera system 108 in a position. For example, the camera system 108 may be implemented in a smartphone and a sensor shield may be provided which includes a slot to receive the smartphone and position the smartphone such that the image sensor and the flash of the smartphone are positioned at an aperture of the box. In some examples, a sensor shield may include one or more filters (e.g., a neutral density filter), diffusers, and/or other light-modifying components between the aperture proximate the camera system and the aperture proximate the subject's eye(s).

Using sensor shields, the image data may accordingly be segmented (e.g., portions of the image data associated with sclera may be extracted) based on pixel offsets associated with a geometry of the sensor shield. For example, consider that the subject's eye may be positioned over an aperture of the sensor shield. The camera system may similarly be positioned over another aperture. In this manner, the camera system and the subject's eye may be positioned in a fixed (e.g., known) position relative to one another. In other examples, other devices may be used to position a camera system and a subject's eye in a fixed position relative to one another. Because the subject's eye is in a known position relative to the camera system, it may also be known which pixels of a resulting image captured by the capture system are likely to contain image data of the eye and/or sclera. Accordingly, pixel offsets (e.g., pixel distances from an edge, center, and/or other location in the image) may be stored based, for example, on a geometry of the sensor shield, and used to extract portions of the image data associated with the eye and/or sclera. In some examples, although not explicitly shown in FIG. 1, in addition to an image of the eye 126, the camera system 108 may obtain image data associated with one or more calibration structures which may be placed proximate the eye 126. For example, the calibration structures may be above, to the side, and/or below the eye 126. In some examples, the calibration structures may be somewhat forward of the eye. For example, the subject may wear one or more frames (e.g., eyewear frames) which include one or more calibration regions. These may be referred to as calibration frames. The calibration frames may be worn by a subject, and the calibration frames may surround at least one eye of the subject when worn by the subject. The calibration frames may include multiple regions of known colors.

The calibration image data, such as calibration data 104, may be associated with portions of the frames. The image data may accordingly be segmented (e.g., portions of the image data associated with the eye(s) and/or sclera may be extracted) by identifying portions of image data within the frames. For example, the computer system 106 may segment the image data 102 at least in part based on a location of calibration frames represented in the image data 102. For example, the instructions for segmenting image data 118 of FIG. 1 may include instructions for locating portions of the image data associated with the known shape and/or color of the frames. The regions of interest may be identified, at least initially, as regions bounded by the frames (e.g., where the eye(s) may be expected to be).

In some examples, to perform color calibration, the image data may be segmented to extract portions of the image data associated with calibration structures (e.g., calibration data 104). The calibration data 104 may be used to adjust (e.g., calibrate) the image data 102.

During operation, image data associated with an eye of a subject may be generated. For example, a camera system (e.g., a smartphone camera) may be used to capture image data associated with one or more eyes. In some examples, image data associated with one eye of a subject may be captured. In some examples, image data associated with multiple eyes (e.g., two eyes) of a subject may be captured. In some examples, calibration image data may be obtained at the same time (or at a different time) the image data associated with the eye(s) is obtained. For example, one or more calibration structures may be placed proximate the eye(s) when the image data is being captured.

In some examples when a sensor shield is used, a flash of a camera system used to generate the image data may be turned on prior to insertion in the sensor shield, and/or prior to or during image acquisition. In examples using the sensor shield, a flash of the camera system may be the only light source used to illuminate the subject's eyes and/or sclera. In examples when calibration frames are used, camera system flashes may or may not be used prior to and/or during image data acquisition. In some examples, use of a camera system flash may alleviate poor ambient lighting conditions and/or shadows generated at least partially by the calibration frames on a subject's face and/or eyes.

In some examples, image data 102 may be obtained from a single image of a subject's eyes. In other examples, multiple images may be captured. In some examples, images of the sclera may be captured while a subject is gazing in different directions, which may expose different regions of the sclera for imaging. For example, images may be captured while a user is gazing straight ahead, up, right, and/or left. In some examples, gazing down may be avoided due to obscuring the sclera with an eyelid, however in some examples images may additionally or instead be captured while a subject is gazing down. In some examples, a subject's eyelid may be manipulated and/or moved out of the way during the acquisition of images during a downward gaze.

During image capture, the camera system may be any of a variety of distances from the subject's eyes. In some examples, the camera system may be about 0.5 m from the subject's eyes. In some example, the camera system may be less than 1 m from the subject's eyes. In some examples, the camera system may be less than 0.75 m from the subject's eyes. In some examples, the camera system may be less than 0.5 m from the subject's eyes. In some examples, the camera system may be less than 0.3 m from one or both of the subject's eyes. In some example, the camera system may be less than 0.2 m from one or both of the subject's eyes. The camera system may be held at a fixed distance by a sensor shield and/or may be held by another user or the subject themselves.

In some examples another person may capture images of the subject's eyes. However, in some examples, the subject themselves may capture the image data (e.g., by taking one or more “selfies”).

In some examples, the image data may be color calibrated, for example, in accordance with instructions for color calibrating which may be included in executable instructions for bilirubin estimation 116. Color calibrating may include, for example, segmenting the image data to extract portions associated with one or more calibration structures (e.g., frames). Regions associated with a known color may be identified and adjustments may be made to the image data in view of the pixel values or other data associated with the region of a known color. For example, the image data may be color calibrated with respect to portions of the image data containing known color values. Color calibrating the image data may result in a set of color calibrated image data. For example, the computer system 106 may generate color calibrated image data based on the image data 102 (e.g., using the calibration data 104). The computer system 106 then utilizes the image data and/or color calibrated image data to generate an estimate of the subject's bilirubin level. The estimate may be generated by segmenting the image data, generating features based on the image data which are representative of sclera color, and analyzing the features using a regression model (e.g., a machine learning model).

Once an estimated bilirubin level has been identified (e.g., using the machine learning model 122), the bilirubin level may be used for a variety of purposes. The bilirubin level may be displayed (e.g., on a display of the computer system 106 of FIG. 1) and/or communicated to another computer system (e.g., the computer system of a care provider). In some examples, an alert, warning, or other information based on the bilirubin level may be displayed or communicated. Any number of users may take action based on the estimated bilirubin level. For example, dosage of a medication may be changed based on the bilirubin level. Treatment may be initiated, halted, and/or adjusted based on the bilirubin level.

For example, systems described herein may be used as a screening tool for any of a variety of diseases having an abnormal bilirubin level and/or change in bilirubin level as a symptom, such as pancreatic cancer, hepatitis, and/or Gilbert's syndrome. Responsive to an estimated bilirubin level provided by the computer system 106 above a threshold, the computer system 106 may provide an indication that the subject should receive further testing for pancreatic cancer or other disease having jaundice as a symptom. Medical care providers may then administer further test and/or diagnostic procedures (e.g., medical imaging) based on the positive screening indication.

In some examples, multiple bilirubin levels may be estimated over time, and a trend in the bilirubin levels of a subject may be used to adjust a medication dose, initiate, stop, and/or modify treatment, or take other action. For example, in treating pancreatic cancer, a stent may be inserted into the common bile duct. The stent may open the duct so that compounds like bilirubin can be broken down again. Systems described herein may monitor a trend in a subject's bilirubin level. After the procedure to insert the stent, it may be expected that the subject's bilirubin level would decrease. If their bilirubin level instead continues to rise, then there may be issues with the stent or the treatment may be ineffective. A care provider may order further imaging of the stent, conduct a follow-up invasive procedure, proscribe medication, or take other action.

In some examples, bilirubin levels may be used to monitor drug toxicity. When a subject is placed on a particular drug regimen, bilirubin levels provided by systems described herein may be monitored over time. If the pattern of bilirubin levels (e.g., increasing bilirubin levels) is indicative of liver disease which may be caused partially or wholly by the drug regimen, a physician may act responsive to the increasing bilirubin levels to change the drug regimen (e.g., change dosing and/or drugs used).

Examples described herein may accordingly extract portions of image data associated with sclera. For example, the computer system 106 of FIG. 1 may extract portions of image data 102 which are associated with the sclera of eye 126. Any of a variety of segmentation algorithms may be used to extract the portions of image data. However, generally, the process used in some examples preferably should not utilize color of the sclera itself to perform the segmentation. Recall a goal of examples described herein is to provide a bilirubin level, and accordingly, the color of subject's sclera should not be assumed to be white. Rather, the sclera color may vary and may, for example, be more yellow in subjects affected by jaundice.

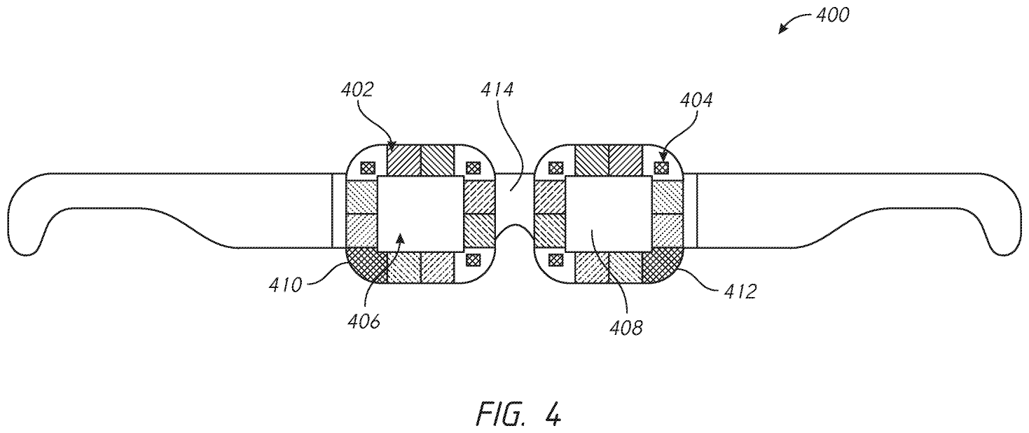

In some examples, a first step in segmenting the sclera from captured image data may be to define regions of interest where the sclera should be located. Some existing methodologies to locate eyes in images may key off of feature around the eyes (e.g., eyebrows). Such methodologies may be inappropriate in some examples described herein where neighboring features may be obscured by a sensor shield and/or calibration frames. In examples utilizing sensor shields, regions of interest may be initially identified as one or more rectangular bounding boxes (e.g., boxes corresponding to images captured from the left and right half side of the sensor shield) using predetermined pixel offsets within the image data. This may be possible because the placement of the camera within the sensor shield is known and the same from image to image. The offsets may be defined such that the regions of interest would cover various face placements and inter-pupillary distances. For example, the boxes used may be large enough to encompass eyes of most subject's given a normal range of face placements and inter-pupillary distances. When calibration frames are used, the regions of interest initially may be defined as one or more regions surrounded by a frame portion (e.g., corresponding to open lens 406 region and open lens 408 region of FIG. 4).

An example sclera segmentation methodology that may be implemented, for example, by the executable instructions for segmenting image data 118, may at least partially utilize the GrabCut method. Multiple (e.g., two) iterations of the method may be used. Generally, GrabCut refers to a methodology for separating a foreground object from its background, where the terms “foreground” and “background” do not necessarily refer to the perceivable foreground and background of the image, but rather a region of interest versus everything else in the image. GrabCut treats the pixels of an image as nodes in a graph. The nodes are connected by edges that are weighted according to the pixels' spatial and chromatic similarity. Nodes in the graph are assigned one of four labels: definitely foreground, definitely background, possibly foreground, and possibly background. After initialization, graph cuts are applied to re-assign node labels such that the energy of the graph is minimized and/or meets some other criteria. Examples described herein utilize segmentation methods (e.g., GrabCut) without human intervention between iterations—e.g., initial bounding boxes may be automatically defined, for example based on sensor shield and/or calibration frame placement, and further iterations may be directed through image analysis techniques.

In examples described herein, a first iteration of the segmentation method (e.g., GrabCut) may learn the color characteristics of the skin and may remove image data regions associated with the skin to isolate the eye. A second iteration may isolate the sclerae by assuming that the scleras are the brightest regions within the eyes (e.g., not necessarily white). Accordingly, the second iteration may utilize brightness, not color profile, to segment the portions of the eye. Generally, then iterations of segmentation methods are described. In each iteration, certain portions (e.g., pixels) of image data may be identified as being “possibly” and/or “definitely” of interest, while certain other portions (e.g., pixels) may be identified as being “possible” and/or “definitely” not of interest.

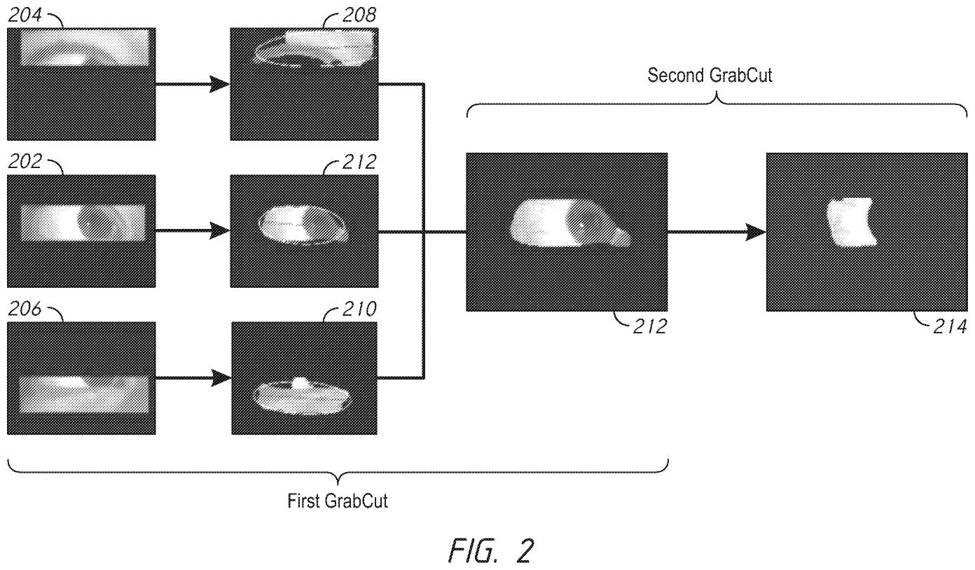

FIG. 2 is a sequence of images illustrating methods of segmenting image data to identify regions associated with sclera arranged in accordance with examples described herein. Image 202, image 204, image 206, image 208, image 210, image 212, and image 214 are shown of a single eye (e.g., eye 126 of FIG. 1) and represent image data during various portions of a segmentation process to identify sclera. Additional, fewer, and/or different images may be used in some examples. Image data generally refers to the data which may be stored and from which an image may be rendered—e.g., any of the images shown in FIG. 2. For example, image data may refer to pixel values used to render the image 202. The actions taken to implement the segmentation described herein and with reference to FIG. 2 may be implemented, for example using computer system 106 of FIG. 1 and executable instructions for bilirubin estimation 116, including executable instructions for segmenting image data 118.

While the images in FIG. 2 illustrate a single eye, multiple eyes may be present in other examples. As described herein, an image of one or more of a subject's eyes may be captured by a camera system. In some examples, before segmentation begins, bilateral filtering may be applied to smooth local noise while maintaining strong edges. An initial bounding box may be applied to limit the image data of interest, resulting in the image 202 showing generally only a region about and including the subject's eye. For example, pixel offsets associated with a sensor shield may be used to define the initial bounding box and/or an area defined by calibration frames may be used to define the initial bounding box. While a bounding box is shown and described as a rectangle, generally the bounding box used to initialize this process may have any shape, including a circle and/or oval in some examples. Generally, the size of the bounding box may vary based on a distance between the camera system and the subject's eye during image acquisition. Accordingly, the executable instructions for segmenting image data 118 may include instructions for defining an initial bounding box, and data outside of the initial bounding box may be discarded and not further processed (e.g., except for calibration data in some examples).

A first iteration of a GrabCut method may extract a region of image data associated with a subject's eye, resulting in image data associated with image 212. This first iteration may not only limit the search space for the sclera, but also removes most of the skin around the eye, reducing effects those pixels could have on color histograms or adaptive thresholds later in the methodology.

In some examples, initial bounding boxes at multiple locations may be tested, an output most likely to contain the most amount of eye is selected. For example, image 204 and image 206 represent different bounding boxes used to initially segment the same image data used to generate image 202, however the bounding boxes are at different locations in each image. The resulting first segmentation iteration from image 204 yields image 208. A first segmentation iteration from image 202 yields image 212. A first segmentation iteration from image 202 yields image 210. Image 208, image 212, and image 210 may then be compared and evaluated to determine which output is most likely to contain only the eye, and accordingly, which bounding box location may be desired. To determine which output is most likely to only contain the eye, the segmented regions from each initialization are evaluated using a variety of metrics. Examples of metrics which may be used to evaluate whether segmented images after a first segmentation iteration contain mostly eye include area fraction—the fraction of the region's area over the total region of interest (e.g., the area represented by the output of the first segmentation versus the input bounding box area). It may be desirable for the area fraction metric to be minimized to indicate a better initialization. Another example metric which may be used to evaluate whether segmented images after a first segmentation iteration contain mostly eye is ellipse area fraction, which refers to the fraction of the region's area over an ellipse area that best fits the output region. It may be desirable for the ellipse area fraction to be maximized to indicate a better initialization. Another example metric which may be used to evaluate whether segmented images after a first segmentation iteration contain mostly eye is incline, which refers to the incline of an ellipse that best fits the output region. It may be desirable for the incline metric to be minimized to indicate a better initialization. Another example metric which may be used to evaluate whether segmented images after a first segmentation iteration contain mostly eye is color variation, which refers to the standard deviation of color across the output region. It may be desirable to maximize the color variation metric to indicate a better initialization. Another example metric which may be used to evaluate whether segmented images after a first segmentation iteration contain mostly eye is variation over borders, which refers to the standard deviation of the brightness values across the top and bottom borders of the bounding box used to initialize the segmentation. It may be desirable to minimize the variation over borders metric to indicate a better initialization. Any combination of these metrics may be used. For example, the described metrics that are desired to be minimized may be negated such that higher values always imply that the region is more eye-like. The metrics may be combined, for example, using the Mahalanobis distance relative to all of the other segmented regions. Overall, this calculation may result in high distances for segmented regions that are small, elliptical, flat, and diverse in color, as well as rectangular initializations that likely do not crop out the eye. The segmented region with the highest distance may be selected and passed along to the second part of the sclera segmentation methodology. For example, best-fit ellipticals and their inclines may be evaluated for image 208, image 212, and image 210. Based on an evaluation of the metrics described, computer system 106 may select image 212 for use by a next stage in the segmentation methodology (e.g., for the second iteration of a GrabCut method).

The executable instructions for segmenting image data 118 may include instructions for defining an initial bounding box, performing at least a first iteration of a segmentation method to extract image data associated with a subject's eye, and performing a second iteration of a segmentation method to extract image data associated with the subject's sclera.

After the first iteration of segmentation to extract a subject's eye, the pixels that are assigned to the foreground in a GrabCut method are considered to be part of the eye, regardless of whether they are labeled as “definitely foreground” or “possibly foreground”. A second iteration of segmentation (e.g., GrabCut) is then used to extract the sclera region from the image data associated with the eye (e.g., to arrive at image 214 of FIG. 2). In some examples, a second iteration of GrabCut may utilize user interaction. In some examples, however, the second iteration may proceed automatically using adaptive and pre-defined thresholds. For example, after converting image data representing image 212 to the HSL color space, four possible pixel assignments are initialized as follows:
Definitely foreground: Top 90th-percentile of L channel values
Definitely background: Bottom 50th-percentile of L channel values
Possibly foreground: Otsu threshold on L channel values
Possibly background: Inverse Otsu threshold on L channel values

Other thresholds may be utilized in other examples. For example, definitely foreground may be top 80th percentile of L channel values in some examples, top 95th percentile of L channel values in some examples. Definitely background may be bottom 40th-percentile of L channel values in some examples, bottom 30th percentile of L channel values in some examples. Possibly foreground and background values may be selected by sorting pixels by another threshold other than the Otsu threshold and inverse (e.g., above and below a threshold value for brightness and/or color may be used). In cases when a pixel satisfies multiple assignments, the strongest assertion may be prioritized (e.g., definitely foreground may be selected over possibly foreground). These assignments are based on the assumption that the brightest region in the eye should be the sclera. This assumption may not hold when glare appears within the eye, as may occur with use of sensor shields and/or calibration frames. Glare corresponds to high values in the lightness channel of the HSL image (L>230). Pixels with glare are accordingly replaced and/or removed in some examples. For example, inpainting may be used. Inpainting refers to a reconstruction process that re-evaluates glare pixels' values via the interpolation of nearby pixels. Once a second iteration of the segmentation method is performed, the pixels that belong to the “definitely foreground” and “possibly foreground” labels are selected. The resulting mask may then be cleaned by a morphological close operation to remove any tiny regions.

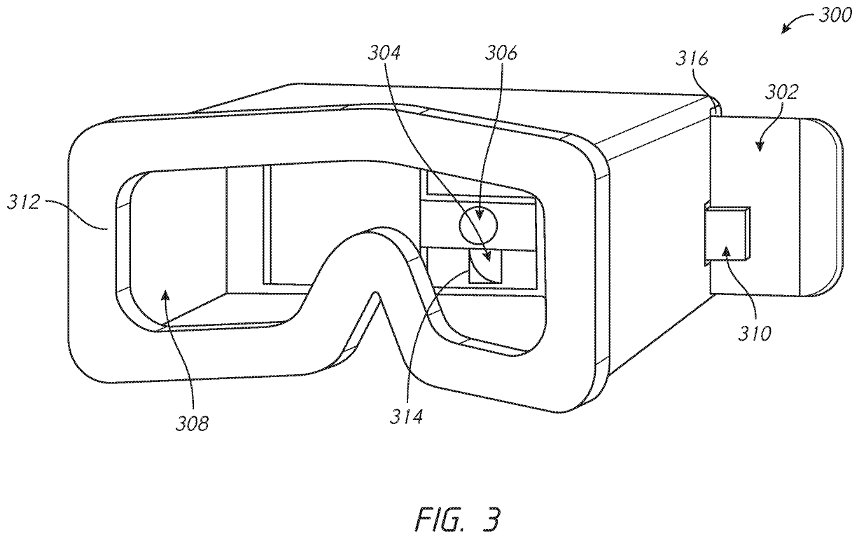

FIG. 3 is a schematic illustration of a sensor shield arranged in accordance with examples described herein. The sensor shield 300 includes box 308, filter holder 310, aperture 312, aperture 314, and slot 316. The sensor shield 300 may interface with smartphone 302 having a camera system (e.g., image sensor 304 and flash 306). Additional, fewer, and/or different components may be used in other examples.

The box 308 at least partially defines aperture 312 and aperture 314. The box 308 may be made of any of a variety of materials, and in some examples may partially and/or wholly opaque to aid in the blocking and/or reduction of ambient light incident on an image sensor of the smartphone 302. In some examples, box 308 may be 3D printed. In some examples box 308 may be implemented using cardboard.

The aperture 312 is sized and positioned to receive one or more eyes of a subject. For example, the face and/or eyes of a subject may be pressed against box 308 such that the eyes are proximate (e.g., over, optically exposed to) the aperture 312. In the example of FIG. 3, the aperture 312 is sized to receive both eyes of a subject. In other examples, the aperture 312 may be sized to receive only a single eye and/or two apertures may be provided, one to receive each eye of a subject.

The aperture 314 is sized and positioned to receive a camera system and allow the camera system to image the eyes of the subject through the aperture 312. For example, a camera system of the smartphone 302 may be positioned proximate (e.g., over, optically exposed to) the aperture 314. In this manner, a camera system may be placed in a fixed spatial relationship with one or more eyes of a subject, and ambient illumination may be blocked and/or reduced from being incident on the camera system.

In some examples, one or more filters, diffusers, and/or other optically modifying components may be included in box 308. As shown in FIG. 3, a filter holder 310 is provided which may span all or a portion of the aperture 314 and hold a filter between the aperture 314 and aperture 312 (e.g., in use, between a camera system and one or more eyes of a subject). The filter may be implemented using a neutral density filter. A neutral density filter and/or a diffuser may, for example, soften illumination from a flash of the smartphone 302 and improve the experience for the subject, who may otherwise find illumination from the flash too harsh.

The box 308 may include and/or be coupled to slot 316 which may receive the camera system (e.g., the smartphone 302). The slot 316 may be implemented, for example, using a rectangular channel. The slot 316 may urge the smartphone 302 against the box 308 and aperture 314. The slot 316 may fix the placement of the smartphone 302 relative to the subject's face by, for example, centering the phone's camera system and maintaining it at a fixed distance.

Note that, in some examples, there may be no electrical connection between box 308 and the smartphone 302. Generally, the box blocks out and/or reduces ambient lighting while allowing the camera system flash to provide illumination (which may be the only illumination) onto the subject's eye(s). Note that physics-based models for color information typically consider an object's visible color to be the combination of two components: a body reflection component, which describes the object's color, and a surface reflection component, which describes the incident illuminant. When using digital photography, color information that gets stored in image files may be impacted by the camera sensor's response to different wavelengths. In the example of FIG. 3, by using the flash 306 as the only illumination source on the sclera, the surface reflection component may be kept constant for all images. This leaves the body reflection component and the camera sensor's response as the only two components that may significantly affect the sclera's appearance. Images of multiple subjects may be captured using the same device, holding the camera sensor's response constant and leaving the body reflection component as a variable. Accordingly, variation between image data captured for multiple subjects may represent the body reflection differences from the subjects' sclera.

FIG. 4 is a schematic illustration of calibration frames arranged in accordance with examples described herein. The calibration frames 400 include region of known color 402, fiducial 404, frame portion 410, frame portion 412, open lens 406, open lens 408, and reference portion 414. Additional, fewer, and/or different components may be used in other examples. The calibration frames 400 are intended to be worn by a user, in a manner analogous to eyewear frames. Frame portion 410 and frame portion 412 define open lens 406 and open lens 408, respectively. Each of frame portion 410 and frame portion 412 may include multiple regions of known colors, such as region of known color 402 in frame portion 410. Each of frame portion 410 and frame portion 412 may include multiple fiducials, such as fiducial 404 of frame portion 412. In use, the calibration frames 400 would be worn by a subject, and the open lens 406 and open lens 408 would be in front of each of the subject's eyes, respectively. The frame portion 410 and frame portion 412 would accordingly be positioned to surround each of the user's eyes.

While the frame portion 410 and frame portion 412 are shown as complete squares that may encircle each of a subject's eyes during use in FIG. 4, other shapes are possible. In some examples, the frame portions may be only a portion of a closed shape (e.g., an arc or partial square may be provided). While two frame portions are shown in FIG. 4, one corresponding to each of a subject's eyes, in some examples, only one frame portion, or more than two frame portions may be provided.

The reference portion 414 is provided between frame portion 410 and frame portion 412 (e.g., on or around a bridge of the subject's nose during use). In other examples, reference portion(s) may be provided in other locations. Generally, the reference portion 414 may be provided with a known color (e.g., black or white) and may be used to aid in locating image data associated with the calibration frames during segmentation.

Open lens 406 and open lens 408 refer to open regions that may allow for the subject's eyes to be imaged through and/or together with the calibration frames. In some examples, open lens 406 and/or open lens 408 may be provided with one or more filters, diffusers, and/or other structures.

Accordingly, calibration frames may be provided having one or more frame portions. Each frame portion may include multiple regions of known color, such as region of known color 402 in FIG. 4.

Calibration frames may be provided having one or more fiducials in addition to and/or instead of multiple regions of known color, such as fiducial 404 in FIG. 4. In the example of calibration frames 400, the upper two corners of each frame portion include a fiducial mark, and the inner (toward the nose when worn) bottom corner of each frame portion also includes a fiducial mark. The arrangement and number of fiducials may vary. Generally, fiducial markers may be placed at one or more corners of frame portions. The fiducials may aid in identifying image data regions associated with the calibration frames 400. For example, the computer system 106 of FIG. 1 may identify fiducial marks in the image data 102, and based on a location of the fiducial marks, may be able to segment the portions of the image data 102 associated with the calibration frames 400 (e.g., calibration data 104). Any of a variety of marks may be used to implement the fiducial marks. In the example of FIG. 4, fiducial 404 is illustrated as a black square surrounded by a larger white square. Generally, any other marking may be used as a fiducial, including a cross, circle, bar code, QR code, striped region, or combinations thereof.

Each frame portion may include multiple regions of known color. In some examples, when multiple frame portions are used, each frame portion may include a same layout and arrangement of regions of known color. In some examples, such as shown in FIG. 4, each of two frame portions may include a mirror image arrangement of regions of known color. For example, the same known colors may be placed closest the nose in each frame portion. In some examples, each of multiple frame portions may include a different arrangement of regions of known color. Colors of the multiple regions of known color used in a frame portion may be selected to aid in calibrating the resulting image data. In the example of FIG. 4, each frame portion includes regions of the following colors: cyan, magenta, yellow, 17% gray, 33% gray, 50% gray, 67% gray, 83% gray, and black. In some examples, colors may be used which are found on color calibration target cards such as the Macbeth ColorChecker. In some examples, a frame portion may include one region of known cyan color, one region of known magenta color, one region of known yellow color, and one known black region. In some examples, a frame portion may additionally or instead include multiple gray regions of varying saturation. In some examples, a frame portion may include one region of red color, one region of blue color, and one region of green color. In some examples, a frame portion may additionally or instead include a region of black color and a region of white color.

In this manner, rather than keeping the surface reflection component and the camera sensor's response constant, image data associated with the regions of known color may allow for images to be normalized to the reference regions. Because the colors of the regions of known color are known, their body reflection component is known and any deviation between their appearance in image data captured by a camera system and their true color may be due to the surface reflection component and the camera system's response.

Accordingly, a calibration matrix may be used to color calibrate image data described herein. The calibration matrix may be based on calibration data which may be associated with images of calibration frames. The calibration matrix may simulate the effects of the color information components associated with the surface reflection component and the camera system's response. The calibration matrix can be applied to the image data associated with the sclerae themselves to reveal a closer estimate of their body reflection component.

During operation, a camera system may capture one or more images of a subject's face and/or eyes, for example to obtain image data 102 of FIG. 1. As described herein, the image data 102 may be segmented to extract portions of the image data 102 which are associated with the subject's sclera. In some examples, the image data may include image data associated with calibration frames or other calibration structures (such as when the image data was acquired while a subject was wearing the calibration frames). Examples described herein may perform segmentation to extract calibration data from the image data (e.g., to extract calibration data 104 from image data 102 of FIG. 1). The calibration data 104 may include data associated with calibration frames worn by a subject. Accordingly, the executable instructions for bilirubin estimation 116 may include executable instructions for segmenting calibration data to allow the computer system 106 to perform the calibration data segmentation described herein.

When calibration frames are worn, a goal of the calibration data segmentation may be to identify the borders of the colored squares around the frame portions of the calibration frames and/or the reference portion so that the regions of known color can be located and used for color calibration.

In some examples, calibration data segmentation may include identifying one or more fiducials, such as fiducial 404 of FIG. 4. For example, the fiducial marks shown in FIG. 4 are square-shaped. In some examples, in the image data, the fiducials may have been captured from an angle and may appear more as quadrilaterals. Black quadrilaterals may be found by converting the image data to grayscale and filtering the image data so that only the contours with four corners and a brightness value less than a threshold (e.g., 60) are kept. The small quadrilaterals may correspond to the fiducials, while the others may correspond to the outlines of the regions of known color around the frame portions. Accordingly, fiducials may be used which are significantly smaller (e.g., one-fourth the size or less, one-third the size or less, one-half the size or less) than the regions of known color around the frame portions. Once quadrilaterals are identified, those which are less than a threshold (e.g., half) of the average quadrilateral area are classified (e.g., by computer system 106 of FIG. 1) as fiducials; the other quadrilaterals are classified as regions of known colors. To confirm that the fiducials belong to the calibration frames and not something in the background, pixels outside the border of the identified fiducials may be checked (e.g., by computer system 106 of FIG. 1) to see if they are white.

If any fiducials are not found because of glare or some other error, their locations may be interpolated or extrapolated based on the locations of the discovered fiducial marks and the known geometry of the calibration frames. For example, when there are known fiducials that are along the same vertical and horizontal axes as where the missing fiducial should be, the corners of the missing fiducial can be estimated by using the intersections of those lines. If there are not enough known fiducials to use interpolation, the known relative dimensions of the calibration frames may be used to estimate the fiducial position.

The positions of the fiducials may be used (e.g., by computer system 106 of FIG. 1) to check the positions of the regions of known color. The relationship between the fiducial locations and the regions of known color may be known. For example, referring to the calibration frames 400 of FIG. 4, the regions of known color are provided along straight vertical and/or horizontal lines between the fiducial marks at the corners. Other patterns of relationship between the fiducials and the regions of known color may be used in other examples. Accordingly, one the fiducials are located, the computer system 106 of FIG. 1 may identify guides (e.g., paths such as straight lines) for locating the regions of known color based on their known positional relationship with the fiducials. For example, the fiducials may be connected with straight lines to provide guides for where the other regions should lie. Any quadrilaterals (or other shape when the regions of known color are other shapes) found outside of those bounds may be discarded as the background. The fiducials may then be used to develop a one-to-one mapping between the names of the colored squares (e.g., left yellow, right 33% gray) and their locations in the image. In the end, in the example of FIG. 4, there should be two colored squares along each side of the lenses and black patches at the far bottom corners. The locations of the larger black-bordered quadrilaterals are compared to the expected positions of the colored squares. If the distance between a detected quadrilateral and the expected position of a colored square is less than a quarter of the expected square's width, the quadrilateral is matched with the corresponding label. There may not be enough detected black-bordered quadrilaterals to assign a border to every square label. This can be attributed to, among other reasons, glare from the camera or ambient lighting that obscures black outlines. Like the missing fiducials, the missing colored squares can be found using a combination of interpolation and extrapolation. After the squares around the rims of the glasses are found, the reference portion 414 that rests on top of the bridge of the nose may be selected using a specified offset from the frame portions to provide a color reference (e.g., white color reference). Accordingly, examples of calibration data segmentation may proceed by identifying fiducials, locating expected locations for the regions of known color based on the fiducial locations, and then identifying the regions of known color based on their shape and position.

In some examples, interpolation and extrapolation may proceed assuming the quadrilaterals (e.g., squares) are linearly arranged around the calibration frames. For example, the calibration frames 400 of FIG. 4 may have such a linear arrangement which may make interpolation and extrapolation straightforward. However, in other examples, such a linear relationship may not be expected or used, and other relationships may be used. For example, calibration frames may be bent to fit to a subject's face, which may alter the path between fiducials and regions of known colors. In other examples, regions of known color may be provided of different shapes.

In some examples, regions of a same known color may be provided in multiple frame portions (e.g., in frame portion 410 and frame portion 412). If a particular region of known color associated with one of the frame portions cannot be extracted from the image data, region of known color associated with the other frame portion may instead be used. In this manner, providing duplicative regions of known color on multiple frame portions may aid in the robustness of the color calibration.

The regions of known color may be used to generate a calibration matrix which may be used to color calibrate the image data. Accordingly, the computer system 106 may include executable instructions for generating a calibration matrix and/or performing color calibration of the image data 102. Color calibrating the image data may remove and/or reduce the effects of the ambient lighting and the camera sensor's response, both of which can change the appearance of the sclera and/or the ability for systems to recognize the sclera or provide a bilirubin level based on the sclera region color.

Color calibration generally involves identifying the calibration matrix C that maps the colors in the image data associated with the regions of known color on the calibration frames with their known colors. Mathematically, consider O as the matrix of observed colors and T as the matrix of target (e.g., known) colors, where each row contains an RGB vector (or other color space vector) that corresponds to a colored square. The matrix C defines the linear transform such that:

$$[ \begin{matrix}{T}_{R 1} & {T}_{G 1} & {T}_{B 1} \\ {T}_{R 2} & {T}_{G 2} & {T}_{B 2} \\ ⋮ & ⋮ & ⋮ \\ {T}_{R k} & {T}_{Gk} & {T}_{Bk}\end{matrix} ] = [ \begin{matrix}{O}_{R 1} & {O}_{G 1} & {O}_{B 1} \\ {O}_{R 2} & {O}_{G 2} & {O}_{B 2} \\ ⋮ & ⋮ & ⋮ \\ {O}_{R k} & {O}_{Gk} & {O}_{Bk}\end{matrix} ] [ \begin{matrix}{C}_{11} & {C}_{12} & {C}_{13} \\ {C}_{21} & {C}_{22} & {C}_{23} \\ {C}_{31} & {C}_{32} & {C}_{33}\end{matrix} ] \qquad Equation 1$$

In some examples, the image data may be gamma-encoded. Accordingly, gamma correction may be applied to the observed colors from the image so that linear operations on them are also linear. This may be performed by raising the values in O by a constant (e.g., γ=2.2 for standard RGB image files). After a calibration matrix is applied, the gamma correction can be reversed by raising the values of the matrix to 1/γ.

The calibration matrix C may be calculated using an iterative least-squares approach. The calibration matrix may be first initialized under the assumption that the individual color channels are uncorrelated and only require a gain adjustment that would scale the mean value of the observed channel values to their targets:

$$C = [ \begin{matrix}\frac{mean ( {T}_{Ri} )}{mean ( {O}_{Ri} )} & 0 & 0 \\ 0 & \frac{mean ( {T}_{Gi} )}{mean ( {O}_{Gi} )} & 0 \\ 0 & 0 & \frac{mean ( {T}_{Bi} )}{mean ( {O}_{Bi} )}\end{matrix} ] \qquad Equation 2$$

For each iteration, the current calibration matrix is applied to (e.g., multiplied with) the observed colors to produce calibrated colors (e.g., calibrated image data). The colors represented by the rows may be converted to the CIELAB color space so that they can be compared to the targets in T using the CIEDE2000 color error, a standard for quantifying color difference. A new calibration matrix C may be computed that reduces the sum of squared errors and the process repeats until convergence.

In some examples, the rows of the target color matrix T are defined as the expected RGB (or other color space) color vectors of the regions of known color of the calibration frames. The rows of the observed color matrix O may be computed by finding the median vector in the HSL color space (or other color space) of the pixels within the bounds of the calibration data identified as corresponding with the known region during the calibration segmentation, and converting the vector back to RGB. For a region R with N 3-dimensional colors, the median vector is defined as:

$${v}_{m} = {argmin}_{{v}_{i} ∈ R} {\sum }_{j = 1}^{N} {‖ {v}_{i} - {v}_{j} ‖}^{2} \qquad Equation 3$$

The median vector may be preferred over taking the mean or median across the channels independently because it may aid in ensuring that the result is a color that exists within the original image. If the channels were treated independently, the combination of values in the three channels may not ever appear in the image data. The difference between the two approaches is typically insignificant when the region is uniform (as is the case with the colored squares), but is a precaution which may be taken nonetheless.

The color calibration may be performed for both eyes. In some examples, regions of known color associated with each eye are used to perform color calibration of image data associated with that eye. For example, regions of known color of frame portion 410 of FIG. 4 may be used to calibrate image data of an eye appearing in open lens 406, while regions of known color of frame portion 412 may be used to calibrate image data of an eye appearing in open lens 408. However, in some examples, a single set of calibration data may be used to calibrate image data pertaining to both eyes. In other examples, a mix may be used. Generally, it may be helpful to utilize image data from regions of known color local to the particular eye to aid in addressing ambient lighting effects which are not always uniform; there may be a shadow or beam of light that creates a gradient across the face, making one side look slightly different from the other. In some examples, however, image data associated with a particular region of known color may be unusable, such as when the image data associated with the region is washed out by glare from the camera system's flash. In such an instance, data from the corresponding region for the other eye may instead be used. If the error between the image data associated with a region of known color and the expected color is over a threshold units (e.g., 5 units) different than the error between the corresponding region of known color surrounding the other eye and the expected color, one may be discarded and the calibration data associated with the region having less error may be used. In some examples, of all image data associated with all available regions of a particular known color are unusable (e.g., corrupted by glare or other problem), that color may simply be thrown out and not used in the calibration procedure.

Accordingly, examples described herein may generate image data, and may optionally generate color calibrated image data. The image data may be segmented to extract regions of the image data which are associated with one or more sclera of a subject. The color represented by this extracted data, which may have been color calibrated, may be used by one or more machine learning models to estimate a bilirubin level of the subject (e.g., in accordance with executable instructions for bilirubin estimation 116 of FIG. 1).

In some examples, a calibration procedure may additionally or instead be performed to eliminate and/or reduce image data variation due to different camera systems. For example, a calibration procedure could be performed even when using a sensor shield (e.g., by capturing an image of a color calibration card within the sensor shield box). The resulting calibration matrix would may then be stored and applied on all images taken with the same camera system. This calibration may be performed at a factor or other location prior to use of the system, and/or a user could be prompted to perform calibration before using the system. In some examples, regions of known color (e.g., colored squares) such as those used in the calibration frames described herein may be integrated into the sensor shield box such that a separate color calibration card may not be needed.

In order to utilize a machine learning model, features may be generated based on the extracted portions of image data corresponding to the sclera (e.g., in accordance with executable instructions for generating features 120 of FIG. 1). Note that jaundice (e.g., elevated bilirubin) is characterized by yellow discoloration, so the features generated from the image data should be indicative of the color of pixels belonging to the sclera (e.g., the color of the sclera regions of the eye image). The features may be generated by evaluating one or more metrics using the extracted data. In some examples, the metric may be a median color vector of the image data associated with the sclera. Use of the median rather than the mean as a metric may aid in ensuring that the sclera color is accurately represented by the metric. For example, the sclera region may frequently contain other components, such as blood vessels or a gradient from the eye's curvature. In these cases, aggregating color channels independently and/or using a mean metric may lead to a color that is not present in the sclera. For example, if an otherwise pristine sclera contains many blood vessels, taking the mean of the color channels independently may generate a feature representing the color of the sclera as a pinkish color. A median vector may instead reflect a white color (e.g., assuming there is more white area than there is red). The median vector may also be useful for when sclera segmentation includes superfluous pixels outside of the sclera. Assuming most pixels belong to the sclera, those pixels do not factor in to the features and final sclera color representation.

In generating features, two processes are generally conducted. First, a group of image data (e.g., pixels) is selected for use in the feature. In some examples, all image data having been extracted as corresponding to the sclera (e.g., all pixels surviving a segmentation method) may be used to provide features. In some examples, however, portions of the image data, even after surviving segmentation as part of the sclera, may not be used in generating features. For example, the segmentation process may generally extract image data within a boundary of a sclera region. However, not all image data (e.g., pixels) within the boundaries of the sclera may actually represent the color of the sclera. Blood vessels, eyelashes, debris, and/or other structures may be present within the sclera boundary. Moreover, glare may render some image data not representative of true sclera color. Use of a median vector as a feature may alleviate the impact of the image data associated with these non-sclera structures and/or, but as an extra precaution, further pixels may be discarded based on their brightness values.

In some examples, image data (e.g., pixels) corrupted by glare may not be used in a process to generate features. Image data corrupted by glare may be identified as any pixels having brightness greater than a threshold value. For example, pixels having a luminance (L) greater than a threshold value in HSL color space may not be used to generate features. The threshold may vary—in some examples, only pixels having an L less than 220 may be used. In some examples, less than 200. In some examples, less than 240. Other thresholds may be used in other examples.

In some examples, image data (e.g., pixels) associated with blood vessels may not be used in a process to generate features. Image data associated with blood vessels may be identified as any pixels having a hue (H) in HSL color less than a threshold value. For example, pixels having an H less than a threshold value in HSL color space may not be used to generate features. The threshold may vary—in some examples, only pixels having an H of greater than 15 may be used. In some examples, greater than 10. In some examples, greater than 20. Other thresholds may be used in other examples.

In some examples, image data (e.g., pixels) associated with eyelashes may not be used in a process to generate features. Image data associated with eyelashes may be identified as any pixels having a luminance (L) less than a threshold in HSL color space. For example, pixels having an L less than a threshold value in HSL color space may not be used to generate features. The threshold may vary—in some examples, only pixels having an L greater than 5 may be used. In some examples, greater than 10. In some examples, greater than 2. Other thresholds may be used in other examples.

Examples of thresholds for eliminating various problematic pixels may in some examples be set empirically by examining images with prominent cases of glare, vessels, and eyelashes. Accordingly, the thresholds may be user-defined in some examples and may change in different settings.

Accordingly, portions of image data may be discarded from image data representative of the sclera. After segmentation, additional criteria based on the luminance and/or hue of the image data may be used to eliminate particular portions of the pixel data from consideration when generating features. Accordingly, image data may be discarded which may be associated with glare, eyelashes, blood vessels, and/or other debris. The criteria may be evaluated by one or more computer systems—e.g., by computer system 106 of FIG. 1 in accordance with executable instructions for bilirubin estimation 116. Example criteria for identifying glare pixels and/or pixel values representative of other structures (e.g., eyelashes, blood vessels) using HSL color space values have been described, however, other criteria may be used including using values from other color spaces (e.g., RGB) in other examples. As an example of a total combination criteria used to eliminate image data likely associated with glare, eyelashes and blood vessels, pixels may be used in feature generation when they have a luminance between upper and lower thresholds (e.g., no glare or eyelashes) and when they have a hue greater than a lower threshold (e.g., no blood vessels).

In some examples, multiple image data sets may be used to generate features. For example, one set of features may be generated using all pixels surviving the sclera segmentation process. Another set of features may be generated using all pixels surviving the sclera segmentation process with pixels associated with glare removed. Another set of features may be generated using all pixels surviving the sclera segmentation process with pixels associated with glare and eyelashes removed. Another set of features may be generated using all pixels surviving the sclera segmentation process with pixels associated with glare, eyelashes, and blood vessels removed. Other image data sets may be used in other examples to generate features.

Another factor to consider in generating features is which color space to use. Generally, images may be acquired in an RGB color space. Converting image data to a different color space involves a calculation across the three channels that express those numbers in a different way. In some examples transformation into a different color space may be performed (e.g., learned) by one or more machine learning models (such as machine learning model 122). However, in some examples, explicitly carrying out color conversions may rearrange the color data in such a way that fewer features may be used. In some examples, features may be generated in multiple color spaces. Features may be generated in RGB, HSL, HSV, L*a*b, and/or YCrCb color spaces. In one example, a feature generated in each color space may be a median color vector of the image data remaining (e.g., surviving the sclera segmentation process and any discarded pixels associated with glare or other structures). In some examples, a feature generated may be pairwise-ratios of color channels (e.g., pairwise-ratios of the three channels in RGB color space). Generally, a yellower color may be expected to have low blue-to-red and blue-to-green ratios, so features representing pairwise-ratios may be useful in correlating with bilirubin level.

Accordingly, features may be generated by evaluating one or more metrics over one or more image data selection groups and one or more color spaces. Not all of the features may be used by a machine learning model, such as machine learning model 122. Some pixel selection methods across the same regions can result in the same features, and some channels across color spaces represent the same information in similar manners. Automatic feature selection may be used to select the most explanatory features and eliminate redundant ones. A top fraction (e.g., 5% in some examples) of the features that explain the data (e.g., sclera color) according to the mutual information scoring function may be used by the machine learning models. Mutual information generally measures the dependency between two random variables. In some examples, features that best represent the image data (e.g., sclera color) may come from looking at the ratio between the green and blue channels in the RGB color space. Recall a healthy sclera should be white, which generally produces high values across all three color channels. Blue is the opposite of yellow, so as the blue value of a white color is reduced, it becomes more yellow. This means that a high green-to-blue ratio may imply a more jaundiced sclera.

Features may be used by one or more machine learning models (e.g., machine learning model 122 of FIG. 1) to generate an estimated bilirubin level based on the image data. In some examples, the machine learning model used may vary in accordance with the system setup. For example, one machine learning model may be used if a sensor shield is used in the system. Another machine learning model, which may have different parameters (e.g., coefficients, cost functions) may be used if calibration frames are present in the system. Accordingly, the machine learning model used may be particular to the presence of one or more components in a system in some examples. In some examples, the machine learning model used may be particular to a camera system used. For example, multiple machine learning models may be trained, one for each of an expected type of camera system (e.g., smartphone) anticipated for use. In other examples, a calibration procedure may be performed to eliminate and/or reduce variation caused by the use of different camera systems.

Examples of machine learning models include regression models (e.g., random forest regression). Example machine learning models may be trained on sclera images and features generated based on image data of subjects having known bilirubin levels (e.g., through blood testing). In some examples, one or more fully convolutional neural networks (FCNs) may be used to implement a machine learning model. FCNs generally take advantage of regular convolutional networks that have been trained to reach high accuracy at identifying objects, only instead of the fully-connected layers at the end that produce object labels, FCNs may use deconvolutions to achieve a label for every pixel. Such a network may be trained for use as machine learning model 122 of FIG. 1 in some examples.

Generally, multiple images may be acquired (e.g., multiple sets of image data) per subject, including multiple images per eye in some examples. Each image may in some examples be used to generate a separate estimated bilirubin level, and the estimated bilirubin level from multiple images and/or eyes of a subject may be combined (e.g., averaged) to provide a final estimated bilirubin level. In some examples, a subset of images may be selected for use in combining its resulting estimated bilirubin level with others to generate a final estimated bilirubin level.

The estimated bilirubin level may in some examples be expressed as mg/dl and intended to be comparable to levels reported through bilirubin blood testing (e.g., TSB). In some examples, the estimated bilirubin level may be intended to be comparable to levels reported through TcB or other bilirubin reporting method. Accordingly, the machine learning model used may be arranged to convert features into an estimated bilirubin level which is comparable to results obtained through any of a variety of other testing mechanisms.

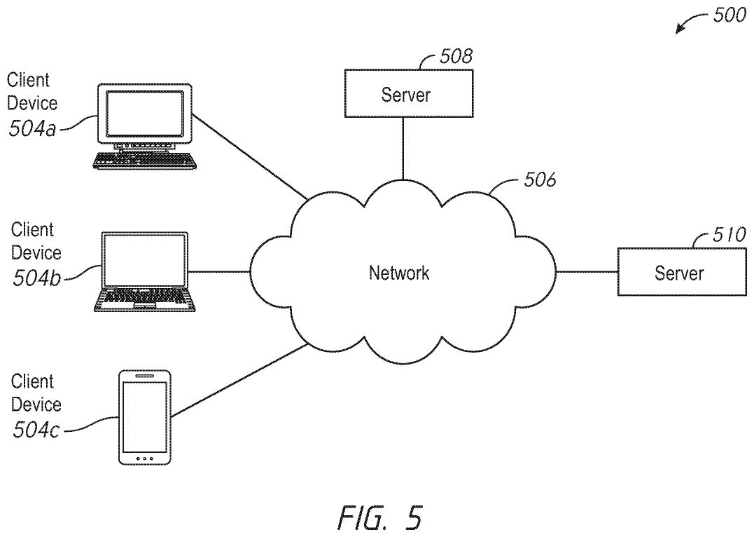

FIG. 5 is a block diagram of example computing network arranged in accordance with an example embodiment. In FIG. 5, servers 508 and 510 are configured to communicate, via a network 506, with client devices 504a, 504b, and 504c. As shown in FIG. 5, client devices can include a personal computer 504a, a laptop computer 504b, and a smart-phone 504c. Other types of client devices may be used in other examples. More generally, client devices 504a-504c (or any additional client devices) can be any sort of computing device, such as a workstation, network terminal, desktop computer, laptop computer, wireless communication device (e.g., a cell phone or smart phone), medical device, appliance, vehicle, and so on. In particular, some or all of client devices 504a-504c can be a source or recipient of data associated with a bilirubin detection device as disclosed herein, as well as the device in which such bilirubin detection is implemented or implemented in part. In many embodiments, clients 504 can perform most or all of the herein-described methods. For example, one or more of clients 504 may be used to implement and/or implemented by computer system 106 of FIG. 1.

The network 506 can correspond to a local area network, a wide area network, a corporate intranet, the public Internet, combinations thereof, or any other type of network(s) configured to provide communication between networked computing devices. In some embodiments, part or all of the communication between networked computing devices can be secured.

Servers 508 and 510 can share content and/or provide content to client devices 504a-504c. As shown in FIG. 5, servers 508 and 510 are not physically at the same location. Alternatively, servers 508 and 510 can be co-located, and/or can be accessible via a network separate from network 506. Although FIG. 5 shows three client devices and two servers, network 506 can service more or fewer than three client devices and/or more or fewer than two servers. In some embodiments, servers 508, 510 can perform some or all of the herein-described methods. For example, servers 508 and/or 510 may be used to implement and/or may be implemented using computer system 106 of FIG. 1.

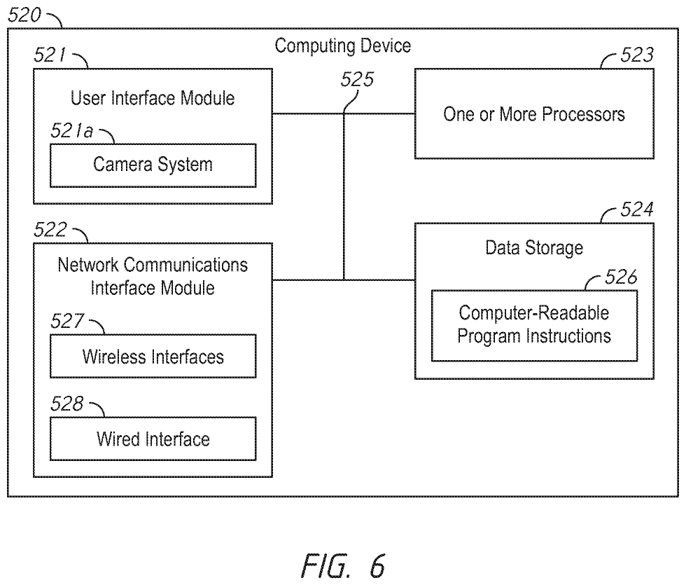

FIG. 6 is a block diagram of an example computing device 520 including user interface module 521, network-communication interface module 522, one or more processors 523, and data storage 524, in accordance with examples described herein.

For example, computing device 520 shown in FIG. 6 can be configured to perform one or more functions of a system, client devices 504a-504c, network 506, and/or servers 508, 510. Computing device 520 may be used to implement the computer system 106 of FIG. 1. Computing device 520 may include a user interface module 521, a network-communication interface module 522, one or more processors 523, and data storage 524, all of which may be linked together via a system bus, network, or other connection mechanism 525.

Computing device 520 can be a desktop computer, laptop or notebook computer, personal data assistant (PDA), mobile phone, video game console, embedded processor, touchless-enabled device, medical device, vehicle, or any similar device that is equipped with at least one processing unit capable of executing machine-language instructions (e.g., executable instructions) that implement at least part of the herein-described techniques and methods (e.g., executable instructions for bilirubin estimation 116 of FIG. 1). In many embodiments, computing device 520 is a smartphone.

User interface 521 can receive input and/or provide output, perhaps to a user. User interface 521 can be configured to send and/or receive data to and/or from user input from input device(s), such as a keyboard, a keypad, a touch screen, a computer mouse, a track ball, a joystick, camera, and/or other similar devices configured to receive input from a user of the computing device 520. In some embodiments, input devices can include gesture-related devices, such a video input device, a motion input device, time-of-flight sensor, RGB camera, or other 3D input device. User interface 521 can be configured to provide output to output display devices, such as one or more cathode ray tubes (CRTs), liquid crystal displays (LCDs), light emitting diodes (LEDs), displays using digital light processing (DLP) technology, printers, light bulbs, and/or other similar devices capable of displaying graphical, textual, and/or numerical information to a user of computing device 520. User interface module 521 can also be configured to generate audible output(s), such as a speaker, speakerjack, audio output port, audio output device, earphones, and/or other similar devices configured to convey sound and/or audible information to a user of computing device 520. As shown in FIG. 6, user interface can be configured with camera system 521a that includes an image sensor and a flash (e.g., camera system 521a may be used to implement and/or may be implemented by camera system 108 of FIG. 1). The light source may include a flash providing the illumination discussed elsewhere herein, while the camera may obtain the video or other image data also discussed elsewhere herein. In embodiments, the functions of the camera system may be performed on a separate and/or remote device in communication with client device 520.

Network-communication interface module 522 can be configured to send and receive data over wireless interface 527 and/or wired interface 528 via a network, such as network 506. Wireless interface 527 if present, can utilize an air interface, such as a Bluetooth®, Wi-Fi®, ZigBee®, and/or WiMAX™ interface to a data network, such as a wide area network (WAN), a local area network (LAN), one or more public data networks (e.g., the Internet), one or more private data networks, or any combination of public and private data networks. Wired interface(s) 528, if present, can comprise a wire, cable, fiber-optic link and/or similar physical connection(s) to a data network, such as a WAN, LAN, one or more public data networks, one or more private data networks, or any combination of such networks.

In some embodiments, network-communication interface module 522 can be configured to provide reliable, secured, and/or authenticated communications. Communications can be made secure (e.g., be encoded or encrypted) and/or decrypted/decoded using one or more cryptographic protocols and/or algorithms, such as, but not limited to, DES, AES, RSA, Diffie-Hellman, and/or DSA. Other cryptographic protocols and/or algorithms can be used as well as or in addition to those listed herein to secure (and then decrypt/decode) communications.

Processor(s) 523 can include one or more central processing units, computer processors, mobile processors, digital signal processors (DSPs), microprocessors, computer chips, and/or other processing units configured to execute machine-language instructions and process data. Processor(s) 523 can be configured to execute computer-readable program instructions 526 that are contained in data storage 524 and/or other instructions as described herein.

Data storage 524 can include one or more physical and/or non-transitory storage devices, such as read-only memory (ROM), random access memory (RAM), removable-disk-drive memory, hard-disk memory, magnetic-tape memory, flash memory, and/or other storage devices. Data storage 524 can include one or more physical and/or non-transitory storage devices with at least enough combined storage capacity to contain computer-readable program instructions 526 and any associated/related data structures.

Computer-readable program instructions 526 and any data structures contained in data storage 526 include computer-readable program instructions executable by processor(s) 523 and any storage required, respectively, to perform at least part of herein-described methods (e.g., executable instructions for bilirubin estimation 116 of FIG. 1).

From the description herein it will be appreciated that, although specific embodiments have been described herein for purposes of illustration, various modifications may be made while remaining with the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signal may be provided directly to and/or received directly from the recited components without intervening components, but also may be provided to and/or received from the certain components through intervening components.

### Implemented Example

An implemented example system was used in a 70-person study including individuals with normal, borderline, and elevated bilirubin levels. An example system utilizing a sensor shield box estimated an individual's bilirubin level with a Pearson correlation coefficient of 0.89 and a mean error of −0.09±2.76 mg/dl when compared to a TSB. An example system utilizing calibration frames provided a Pearson correlation coefficient of 0.78 and a mean error of 0.15±3.55 mg/dl.

Data for the study was collected through a custom app on an iPhone SE. The images collected by the app were at a resolution of 1920×1080. Images were collected utilizing a sensor shield box in one portion of the study, and using calibration frames as described herein in another portion of the study. Before the use of either accessory, the smartphone's flash was turned on. Keeping the flash constantly on rather than bursting it at the time of the pictures was a consideration for participant comfort since the stark change in lighting can be unpleasant. When using the glasses, the flash was left on in case there was insufficient lighting in the room or the glasses created a shadow on the participant's face.

After the flash was turned on, the smartphone was placed in the sensor shield box, such as by inserting it in the slot of the sensor shield schematically illustrated in FIG. 3. A hole in the back of the box provided access to the screen for starting and stopping data collection. The app prompted the participant to look in four different directions—up, left, right, and straight ahead—one at a time while taking a picture after each.

During the portion of the study utilizing calibration frames, the smartphone was held approximately 0.5 m away from the participant's face to take pictures with the calibration frames. This distance is roughly how far away we would expect participants to hold their smartphones if they were taking a selfie.

Each participant looked at each direction for two trials per accessory, yielding 2 accessories×2 trials per accessory×4 gaze directions per trial=16 images per participant.

The smartphone was at a fixed distance of 13.5 cm from the person's face when the sensor shield was in use and at a variable, farther distance when the calibration frames were in use. The size of the rectangle used to initialize the first iteration of GrabCut had fixed dimensions for the sensor shield (˜600×200 px) and dynamic dimensions according to the size of the frames for the calibration frames (˜90% of width×60% of height).

Following optional color calibration, and sclera segmentation, color representations of the sclera was computed using combinations of pixel selection methods and color spaces. Each color has 3 channels. Five pixel selection methods were used (1. all pixels surviving sclera segmentation, 2. #1-glare pixels, 3. #2-eyelash pixels, 4. #2-blood vessel pixels, 5. #1-glare, eyelash, and blood vessel pixels). Five color spaces were also used (RGB, HSL, HSV, L*a*b, and YCrCb), and pair-wise RGB ratios were calculated. This resulted in 5 pixel selection methods×(5 color spaces×3 channels per color space+6 RGB ratios)=105 features per eye. Not all of the features were used in the final model. Some pixel selection methods across the same regions can result in the same pixels, and some channels across color spaces represent the same information in similar manners. Automatic feature selection was used to select the most explanatory features and eliminate redundant ones. The top 5% of the features that explain the data according to the mutual information scoring function were used in the final models.

Separate machine learning models were developed for the two accessories used (e.g., sensor shield and calibration frames). The models used random forest regression and were trained through 10-fold cross-validation across participants. The distribution of bilirubin levels was not evenly distributed; the healthy participants generally had similarly low values within 0.1 mg/dl, while the abnormal patients had a far wider spread. The thresholds used split the participants such that the normal and elevated classes had roughly equal sizes (31 vs. 25). The borderline class was roughly half as large (14). To ensure that the training sets were balanced during cross-validation, splits were assigned using stratified sampling across the three bilirubin level classes. For example, the typical fold for the dataset includes 3 participants with normal bilirubin levels, 1 participant with a borderline bilirubin level, and 3 participants with elevated bilirubin levels.

The data collection procedure resulted in 2 trials per accessory×4 gaze directions per trial=8 images per accessory. Note that each image contains 2 eyes, leading to 16 eye images per accessory. Each eye was summarized with a feature vector that led to its own bilirubin level prediction. The estimates from the 8 images are averaged to produce a final bilirubin level estimate that was reported back to the user.

In some examples, the sclera boundaries were given a priori, and the features generated, and machine learning model used to generate bilirubin level. In such cases, with the optimal segmentation, the Pearson correlation coefficient between the system's predictions and ground truth TSB values were 0.86 with the sensor shield and 0.83 with the calibration frames. With the sensor shield, the system estimated the user's bilirubin level with a mean error of −0.17±2.81 mg/dl. With the calibration frames, the system estimated the user's bilirubin level with a mean error of −0.08±3.10 mg/dl.

In some examples, sclera segmentation techniques described herein were used to extract image data associated with the sclera. The automatically extracted image data was then used to generate estimated bilirubin levels using machine learning models as described herein. In such cases, the Pearson correlation coefficient for image data taken with the calibration frames dropped to 0.78, and the mean error of that model widened to 0.15±3.55 mg/dl. The Pearson correlation coefficient for the sensor shield system rose to 0.89, and the mean error improved to −0.09±2.76 mg/dl.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

1. A method comprising:
extracting portions of image data associated with sclera from image data associated with an eye of a subject;
generating features describing color of the sclera; and
analyzing the features using a regression model to provide a bilirubin estimate for the subject.

2. The method of claim 1 further comprising:
capturing the image data associated with the eye using a smartphone camera.

3. The method of claim 2, further comprising positioning the smartphone camera over an aperture of a sensor shield, the sensor shield having at least one additional aperture positioned over the eye.

4. The method of claim 3, wherein said extracting portions comprises identifying a region of interest containing the sclera using pixel offsets associated with a geometry of the sensor shield.

5. The method of claim 2, further comprising capturing calibration image data in addition to the image data associated with the eye, the calibration image data associated with portions of frames worn proximate the eye.

6. The method of claim 5, wherein said extracting portions comprises identifying a region of interest containing the sclera by identifying the portions of image data within the frames.

7. The method of claim 1, further comprising color calibrating the image data.

8. The method of claim 7, wherein said color calibrating comprises color calibrating with respect to portions of the image data containing known color values.

9. The method of claim 1, wherein said generating features comprises evaluating a metric over multiple pixel selections within the portions of image data.

10. The method of claim 9, wherein the metric comprises median pixel value.

11. The method of claim 9, wherein said generating features further comprises evaluating the metric over multiple color spaces of the portions of image data.

12. The method of claim 11, wherein said generating features further comprises calculating a ratio between channels in at least one of the multiple color spaces.

13. The method of claim 1, wherein the regression model uses random forest regression.

14. The method of claim 1, further comprising initiating or adjusting a medication dose, or initiating or adjusting a treatment regimen, or combinations thereof, based on the bilirubin estimate.

15. A system comprising:
a camera system including an image sensor and a flash;
a sensor shield having a first aperture configured to receive the camera system and at least one second aperture configured to open toward an eye of a subject, the sensor shield configured to block at least a portion of ambient light from an environment in which the subject is positioned from the image sensor; and
a computer system in communication with the camera system, the computer system configured to receive image data from the image sensor and estimate a bilirubin level of the subject at least in part by being configured to:
segment the image data to extract a portion of the image data associated with a sclera of the eye;
generate features representative of a color of the sclera; and
analyze the features using a machine learning model to provide an estimate of the bilirubin level.

16. The system of claim 15, wherein the camera system comprises a smartphone and wherein the sensor shield includes a slot configured to receive the smartphone and position the smartphone such that the image sensor and the flash of the smartphone are positioned at the first aperture.

17. The system of claim 15, wherein the sensor shield comprises a neutral density filter and diffuser positioned between the first aperture and the at least one second aperture.

18. A system comprising:
calibration frames configured to be worn by a subject, the calibration frames configured to surround at least one eye of the subject when worn by the subject, the calibration frames comprising multiple regions of known colors;
a camera system including an image sensor and a flash, the camera system configured to generate image data from the image sensor responsive to illumination of the at least one eye of the subject and the calibration frames with the flash; and
a computer system in communication with the camera system, the computer system configured to receive the image data and estimate a bilirubin level of the subject at least in part by being configured to:
segment the image data to extract a portion of the image data associated with a sclera of the at least one eye;
calibrate the portion of the image data in accordance with another portion of the image data associated with the calibration frames to provide calibrated image data;
generate features representative of a color of the sclera using the calibrated image data; and
analyze the features using a machine learning model to provide the estimate of the bilirubin level.

19. The system of claim 18, wherein the computer system is further configured to segment the image data at least in part based on a location of the calibration frames in the image data.

20. The system of claim 18, wherein the calibration frames comprise eyewear frames.
